(19) United States
(12) Patent Application Publication
(10) Pub. No.: US 2025/0058352 A1
(43) Pub. Date: Feb. 20, 2025

(54) POWER SUPPLY DEVICE FOR PUSH WAVE GENERATION

(71) Applicants: FUJIFILM Healthcare Corporation, Kashiwa-shi (JP); FUJIFILM Corporation, Tokyo (JP)

(72) Inventors: Kosuke Kambara, Chiba (JP); Koji Shimomura, Tokyo (JP)

(21) Appl. No.: 18/787,252

(22) Filed: Jul. 29, 2024

(30) Foreign Application Priority Data
Aug. 17, 2023 (JP) 2023-132749

(51) Int. Cl. B06B 1/02; A61B 8/00; A61B 8/08

(52) U.S. Cl. CPC B06B 1/0215; A61B 8/56; A61B 8/485

(57) ABSTRACT

A power supply device for push wave generation includes a positive electrode power supply and a negative electrode power supply as power supplies that output power supplied to a pulser circuit to which an ultrasound oscillator is connected. A positive electrode capacitor and a negative electrode capacitor accumulate the power supplied from the positive electrode power supply and the negative electrode power supply. A variable constant voltage circuit is provided between the positive electrode capacitor and the pulser circuit, and a variable constant voltage circuit is provided between the negative electrode capacitor and the pulser circuit. The pulser circuit outputs a positive/negative pulse voltage to the ultrasound oscillator as a push wave transmission signal, and the ultrasound oscillator transmits a push wave in response to the push wave transmission signal.

### CROSS REFERENCE TO RELATED APPLICATION

The present application claims priority from Japanese Patent Application No. 2023-132749, filed on Aug. 17, 2023, the content of which is hereby incorporated by reference into this application.

### BACKGROUND OF THE INVENTION

### 1. Field of the Invention

The present disclosure relates to a power supply device for push wave generation, and particularly, to a power supply device for transmitting a push wave from an ultrasound oscillator.

### 2. Description of the Related Art

There is an ultrasound diagnostic apparatus that generates a shear wave in a biological tissue of a subject, measures a propagation velocity of the shear wave, and measures an elastic property of the biological tissue based on the propagation velocity of the shear wave. In this ultrasound diagnostic apparatus, a push wave is transmitted from an ultrasound oscillator or the like to the biological tissue, and the shear wave is excited in the biological tissue by the push wave. By measuring a displacement of each point of the biological tissue due to the shear wave by transmitting and receiving ultrasound, the propagation velocity of the shear wave is measured, and the elastic property of the biological tissue is measured based on the propagation velocity of the shear wave.

JP2016-73452A, JP2015-58251A, and JP2018-191798A disclose circuit configurations for transmitting a push wave from an ultrasound oscillator to a biological tissue.

### SUMMARY OF THE INVENTION

As the circuit configuration for generating the push wave in the ultrasound oscillator, as disclosed in JP2015-58251A, there is a circuit configuration that accumulates an electric charge in a capacitor. The capacitor is charged from a variable voltage power supply via a constant current source, and a transmission signal is output from a transmission/reception unit to the ultrasound oscillator by the energy of the electric charge accumulated in the capacitor. The ultrasound oscillator transmits the push wave in response to the transmission signal.

In a case in which the shear wave is generated in the biological tissue, for example, the transmission signal having a wave train length of several 100 μsec is used for the push wave. During a period in which the push wave is transmitted, the electric charge of the capacitor is decreased, and thus a charging voltage gradually is decreased. As a result, a droop phenomenon may occur in which the magnitude of the push wave to be transmitted is decreased with the decrease in the charging voltage.

An object of the present disclosure is to transmit a push wave having a sufficient magnitude to a biological tissue for elasticity measurement.

An aspect of the present disclosure relates to a power supply device for push wave generation comprising: a power supply that outputs power supplied to a pulse generation circuit to which an ultrasound oscillator is connected; a capacitor that accumulates the power supplied from the power supply; and a constant voltage circuit that is provided between the capacitor and the pulse generation circuit, in which the pulse generation circuit outputs a transmission signal to the ultrasound oscillator, and the ultrasound oscillator transmits a push wave in response to the transmission signal.

In one embodiment, each time the pulse generation circuit outputs the transmission signal to the ultrasound oscillator over a plurality of times, the power supply charges the capacitor with a voltage higher than an output voltage of the constant voltage circuit.

An aspect of the present disclosure relates to a power supply device for push wave generation being attachably and detachably attached to an ultrasound diagnostic apparatus including a first power supply, an ultrasound oscillator, a pulse generation circuit to which the ultrasound oscillator is connected, and a main power supply path that transmits power from the first power supply to the pulse generation circuit in a case in which an ultrasound image is acquired, the power supply device for push wave generation comprising: a second power supply that outputs supply power to the pulse generation circuit; a capacitor that accumulates the supply power; a constant voltage circuit that is provided between the capacitor and the pulse generation circuit; and a current cutoff/adjuster that is provided between the constant voltage circuit and the pulse generation circuit, in which a downstream end of the current cutoff/adjuster is connected to the main power supply path, and the pulse generation circuit outputs a transmission signal to the ultrasound oscillator by using the power transmitted via the current cutoff/adjuster, and the ultrasound oscillator transmits a push wave to a biological tissue by using the transmission signal.

In one embodiment, the main power supply path includes a main power supply path current cutoff/adjuster that is provided between the first power supply and the pulse generation circuit, and the downstream end of the current cutoff/adjuster is connected between the main power supply path current cutoff/adjuster and the pulse generation circuit.

Still another aspect of the present disclosure relates to a power supply device for push wave generation being attachably and detachably attached to an ultrasound diagnostic apparatus including a power supply, an ultrasound oscillator, a pulse generation circuit to which the ultrasound oscillator is connected, and a main power supply path that transmits power from the power supply to the pulse generation circuit in a case in which an ultrasound image is acquired, the power supply device for push wave generation comprising: a capacitor that accumulates the power output from the power supply and supplied to the pulse generation circuit; a constant voltage circuit that is provided between the capacitor and the pulse generation circuit; and a current cutoff/adjuster that is provided between the constant voltage circuit and the pulse generation circuit, in which a downstream end of the current cutoff/adjuster is connected to the main power supply path, and the pulse generation circuit outputs a transmission signal to the ultrasound oscillator by using the power transmitted via the current cutoff/adjuster, and the ultrasound oscillator transmits a push wave to a biological tissue by using the transmission signal.

In one embodiment, the main power supply path includes a main power supply path current cutoff/adjuster that is provided between the power supply and the pulse generation circuit, and the downstream end of the current cutoff/adjuster is connected between the main power supply path current cutoff/adjuster and the pulse generation circuit.

According to the aspect of the present disclosure, it is possible to transmit the push wave having a sufficient magnitude to the biological tissue for the elasticity measurement.

### BRIEF DESCRIPTION OF THE DRAWINGS

FIG. 1 is a diagram showing a configuration of an ultrasound diagnostic apparatus.

FIG. 2 is a diagram showing a power supply device for push wave generation along with a pulser circuit and an ultrasound oscillator.

FIG. 3 is a diagram showing a circuit configuration example of a variable constant voltage circuit.

FIG. 4 is a diagram showing a voltage of each unit of the power supply device for push wave generation.

FIG. 5 is a diagram showing the voltage of each unit of the power supply device for push wave generation.

FIG. 6 is a diagram showing the power supply device for push wave generation along with a power supply device for an analysis operation, the pulser circuit, and the ultrasound oscillator.

FIG. 7 is a diagram showing an external power supply circuit along with the power supply device for an analysis operation, the pulser circuit, and the ultrasound oscillator.

FIG. 8 is a diagram showing the external power supply circuit along with the power supply device for an analysis operation, the pulser circuit, and the ultrasound oscillator.

### DESCRIPTION OF THE PREFERRED EMBODIMENTS

Embodiments of the present disclosure will be described with reference to the accompanying drawings. The same components shown in a plurality of drawings are denoted by the same reference numerals, and the description thereof will be simplified. The terms indicating directions, such as “up”, “down”, “left”, and “right”, in the present specification indicate directions in the drawings. These terms indicating the directions are used for convenience of description, and do not limit a posture in which each component is disposed. The term “voltage” in the present specification indicates a voltage based on a potential of a ground conductor, unless otherwise specified.

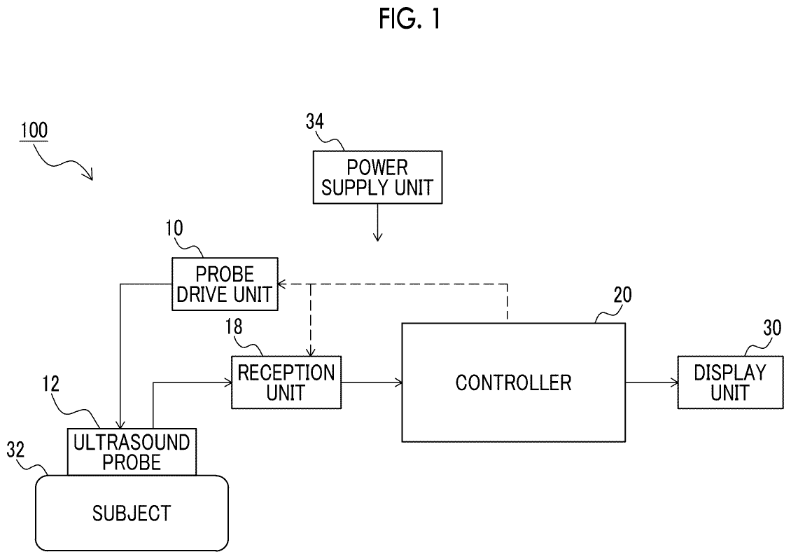

FIG. 1 shows a configuration of an ultrasound diagnostic apparatus 100 according to the embodiment of the present disclosure. The ultrasound diagnostic apparatus 100 comprises a probe drive unit 10, an ultrasound probe 12, a reception unit 18, a controller 20, a display unit 30, and a power supply unit 34. Each of the probe drive unit 10, the ultrasound probe 12, the reception unit 18, the controller 20, the display unit 30, and the power supply unit 34 may be hardware formed by an electric circuit element. The controller 20 may include a processor that executes a program stored in advance. The power supply unit 34 supplies power supply power to the probe drive unit 10, the reception unit 18, the controller 20, and the display unit 30.

The probe drive unit 10 outputs a push wave transmission signal and an analysis wave transmission signal to the ultrasound probe 12 in different time slots under the control of the controller 20. The push wave transmission signal is a signal for transmitting a push wave (ultrasound pulse) to the ultrasound probe 12 in order to excite a shear wave. The analysis wave transmission signal is a signal for transmitting an analysis ultrasound, such as a tracking wave for performing an elasticity analysis and an ultrasound for generating a B-mode image, to the ultrasound probe 12. The ultrasound probe 12 comprises a plurality of ultrasound oscillators. The probe drive unit 10 may output the push wave transmission signal or the analysis wave transmission signal to a necessary oscillator among the plurality of ultrasound oscillators.

The ultrasound probe 12 transmits the push wave to a subject 32 in response to the push wave transmission signal output from the probe drive unit 10, and excites the shear wave in the subject 32. The ultrasound probe 12 transmits the analysis ultrasound for observing a state of a biological tissue of the subject 32 or a propagation state of the shear wave based on the analysis wave transmission signal output from the probe drive unit 10. Each ultrasound oscillator provided in the ultrasound probe 12 receives a reflected ultrasound generated by the reflection in the subject 32 and converts the reflected ultrasound into a reception signal, which is an electrical signal, and outputs the reception signal to the reception unit 18.

The reception unit 18 generates an analysis signal by performing synthesis processing such as phasing addition on the reception signal output from each ultrasound oscillator, to output the analysis signal to the controller 20, in accordance with the control of the controller 20. The controller 20 generates elasticity image data indicating an elastic property based on the analysis signal, and displays the elasticity image based on the elasticity image data on the display unit 30. The elasticity image may be, for example, an image in which color indicating the hardness of the biological tissue is added to the B-mode image.

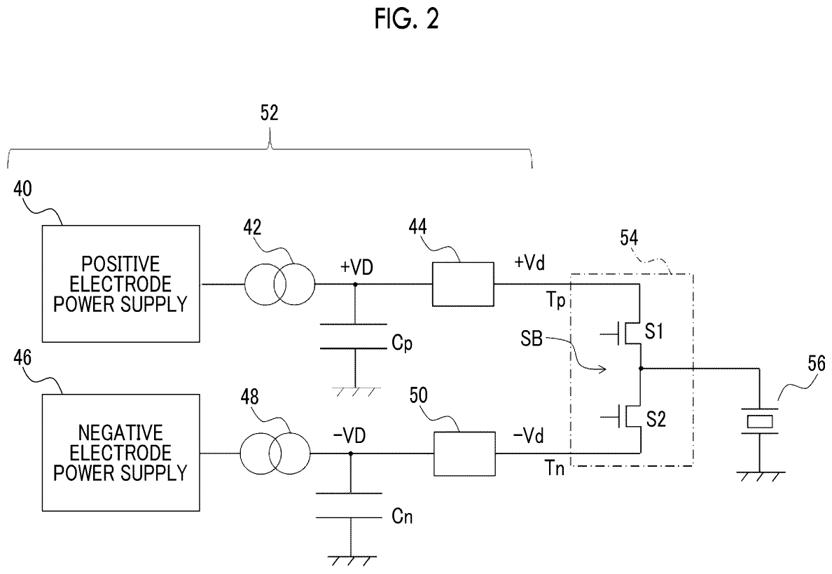

FIG. 2 shows a power supply device for push wave generation 52 included in the ultrasound diagnostic apparatus 100, along with a pulser circuit 54 and an ultrasound oscillator 56. The ultrasound oscillator 56 shown in FIG. 2 is one of the plurality of ultrasound oscillators provided in the ultrasound probe 12 of FIG. 1. The power supply device for push wave generation 52 may be formed in the power supply unit 34 of FIG. 1. The power supply device for push wave generation 52 comprises a positive electrode power supply 40, a constant current circuit 42, a positive electrode capacitor Cp, a variable constant voltage circuit 44, a negative electrode power supply 46, a constant current circuit 48, a negative electrode capacitor Cn, and a variable constant voltage circuit 50. An output terminal of the positive electrode power supply 40 is connected to an upper end of the positive electrode capacitor Cp via the constant current circuit 42. A lower end of the positive electrode capacitor Cp is connected to the ground conductor. The upper end of the positive electrode capacitor Cp is further connected to a positive electrode terminal Tp of the pulser circuit 54 via the variable constant voltage circuit 44.

An output terminal of the negative electrode power supply 46 is connected to an upper end of the negative electrode capacitor Cn via the constant current circuit 48. A lower end of the negative electrode capacitor Cn is connected to the ground conductor. The upper end of the negative electrode capacitor Cn is further connected to a negative electrode terminal Tn of the pulser circuit 54 via the variable constant voltage circuit 50.

The pulser circuit 54 is a pulse generation circuit comprising a switching bridge SB including two switching elements S1 and S2. The two switching elements S1 and S2 are connected in series. In the switching bridge SB, an upper side terminal of the upper side switching element S1 is the positive electrode terminal Tp, and a lower side terminal of the lower side switching element S2 is the negative electrode terminal Tn. A bipolar transistor or a field effect transistor may be used as the switching elements S1 and S2.

In a case in which two bipolar transistors are used as the switching elements S1 and S2, a potential of a base of each bipolar transistor is controlled by the controller 20, so that each bipolar transistor is turned on or off. In a case in which two field effect transistors are used as the switching elements S1 and S2, a potential of a gate of each field effect transistor is controlled by the controller 20, so that each field effect transistor is turned on or off.

The ultrasound oscillator 56 is connected between a connection point between the upper side switching element S1 and the lower side switching element S2, and the ground conductor. As described above, the ultrasound probe 12 includes the plurality of ultrasound oscillators. A plurality of switching bridges SB corresponding to the plurality of ultrasound oscillators are connected parallel, and the ultrasound oscillator is connected to each switching bridge SB. Here, the operation of the power supply device for push wave generation 52 is shown with one ultrasound oscillator 56 among the plurality of ultrasound oscillators provided in the ultrasound diagnostic apparatus 100.

The positive electrode power supply 40 outputs a positive constant voltage to the potential of the ground conductor to the constant current circuit 42. The constant current circuit 42 outputs a current to the positive electrode capacitor Cp and the variable constant voltage circuit 44. The negative electrode power supply 46 outputs a negative constant voltage to the potential of the ground conductor to the constant current circuit 48. The constant current circuit 48 outputs a current to the negative electrode capacitor Cn and the variable constant voltage circuit 50.

The controller 20 (FIG. 1) determines target voltages Vd* and −Vd* output from the variable constant voltage circuit 44 and the variable constant voltage circuit 50, respectively, in accordance with the conditions such as the magnitude of the push wave transmitted from the ultrasound oscillator 56, and controls the variable constant voltage circuit 44 and the variable constant voltage circuit 50.

The variable constant voltage circuit 44 outputs a positive voltage Vd with respect to the potential of the ground conductor to the positive electrode terminal Tp of the pulser circuit 54 based on a terminal-to-terminal voltage VD of the positive electrode capacitor Cp in accordance with the control of the controller 20. The variable constant voltage circuit 50 outputs a negative voltage −Vd with respect to the potential of the ground conductor to the negative electrode terminal Tn of the pulser circuit 54 based on a terminal-to-terminal voltage −VD of the negative electrode capacitor Cn in accordance with the control of the controller 20.

The switching bridge SB provided in the pulser circuit 54 is switched in accordance with the control of the controller 20. For example, the controller 20 turns on the upper side switching element S1 for a predetermined time while maintaining the lower side switching element S2 off, and then turns on the lower side switching element S2 for a predetermined time while maintaining the upper side switching element S1 off, so that a pulse voltage that swings positive and negative (positive/negative pulse voltage) is applied to the ultrasound oscillator 56 as the push wave transmission signal. As a result, the push wave is transmitted from the ultrasound oscillator 56 to the subject 32.

As described above, the power supply device for push wave generation 52 comprises the positive electrode power supply 40 and the negative electrode power supply 46 as the power supplies that output the power supplied to the pulser circuit 54 (pulse generation circuit) to which the ultrasound oscillator 56 is connected. The power supply device for push wave generation 52 further comprises the positive electrode capacitor Cp and the negative electrode capacitor Cn as the capacitors that accumulate the power supplied from the positive electrode power supply 40 and the negative electrode power supply 46. The power supply device for push wave generation 52 further comprises the variable constant voltage circuit 44 (constant voltage circuit) provided between the positive electrode capacitor Cp and the pulser circuit 54, and the variable constant voltage circuit 50 (constant voltage circuit) provided between the negative electrode capacitor Cn and the pulser circuit 54. The pulser circuit 54 outputs the positive/negative pulse voltage to the ultrasound oscillator 56 as the push wave transmission signal, and the ultrasound oscillator 56 transmits the push wave in response to the push wave transmission signal.

In a case in which the elasticity analysis is performed, for example, a continuous transmission operation in which the push waves are continuously transmitted to the subject 32 over a plurality of times is repeated at a predetermined transmission period T. In this case, the controller 20 performs switching control on the switching bridge SB to repeat the switching of applying the positive/negative pulse voltage to the ultrasound oscillator 56 over a plurality of times at the predetermined transmission period T.

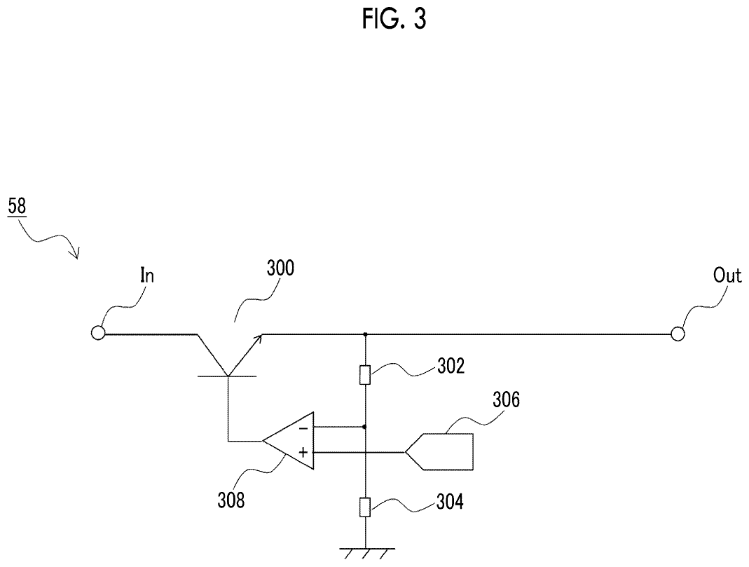

FIG. 3 shows a circuit configuration example of a variable constant voltage circuit 58 used as the variable constant voltage circuit 44 and the variable constant voltage circuit 50. The variable constant voltage circuit 58 comprises a transistor 300, a first voltage division resistor 302, a second voltage division resistor 304, a comparator 308, and a control terminal 306. An output terminal of the comparator 308 is connected to a base of the transistor 300. The first voltage division resistor 302 and the second voltage division resistor 304 are connected in series, and are connected between an output terminal Out and the ground conductor. A voltage division point, which is a connection point between the first voltage division resistor 302 and the second voltage division resistor 304, is connected to a negative electrode terminal of the comparator 308. The control terminal 306 is connected to a positive electrode terminal of the comparator 308.

The comparator 308 outputs a voltage to the base of the transistor 300 in accordance with a difference voltage obtained by subtracting a voltage at the voltage division point between the first voltage division resistor 302 and the second voltage division resistor 304 from a control voltage applied to the control terminal 306. The transistor 300 causes a current to flow from an input terminal In to the output terminal Out in accordance with a base voltage determined by the output voltage of the comparator 308. The controller 20 outputs the control voltage to the control terminal 306. The controller 20 adjusts a voltage of the output terminal Out by changing the control voltage.

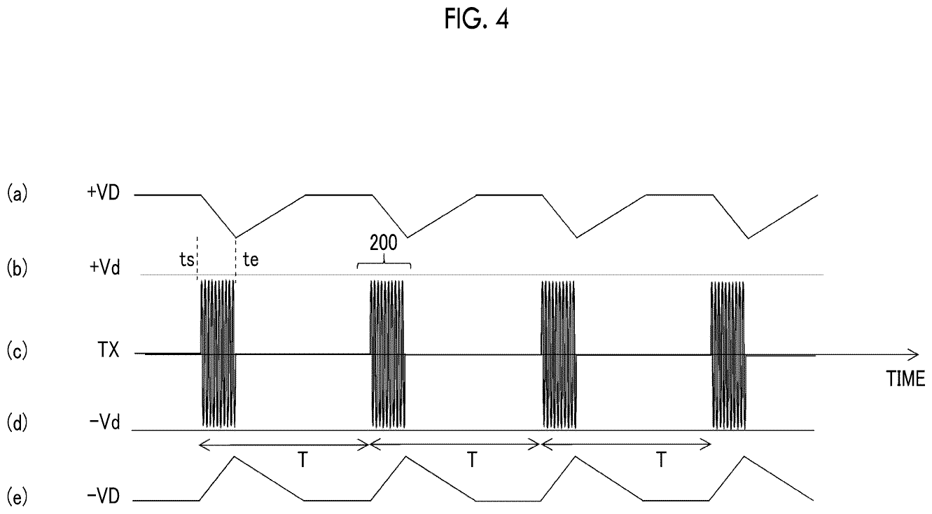

(a) to (e) of FIG. 4 respectively show time waveforms of the terminal-to-terminal voltage VD of the positive electrode capacitor Cp, the voltage Vd of the positive electrode terminal Tp of the pulser circuit 54, a push wave transmission signal TX applied from the pulser circuit 54 to the ultrasound oscillator 56, the voltage −Vd of the negative electrode terminal Tn of the pulser circuit 54, and the terminal-to-terminal voltage −VD of the negative electrode capacitor Cn.

The push wave transmission signal TX is a voltage in which the continuous positive/negative pulse voltage 200 continuously connected on a time axis is repeated at the transmission period T. Each time the continuous positive/negative pulse voltage 200 is applied to the ultrasound oscillator 56, a current flows to the positive electrode terminal Tp of the pulser circuit 54 via the variable constant voltage circuit 44 based on an electric charge accumulated in the positive electrode capacitor Cp. In addition, each time the continuous positive/negative pulse voltage 200 is applied to the ultrasound oscillator 56, a current flows from the negative electrode terminal Tn of the pulser circuit 54 to the negative electrode capacitor Cn via the variable constant voltage circuit 50 based on an electric charge accumulated in the negative electrode capacitor Cn. As a result, the terminal-to-terminal voltage VD of the positive electrode capacitor Cp is reduced from a start time ts to an end time te of the continuous positive/negative pulse voltage 200, and the magnitude of the terminal-to-terminal voltage −VD of the negative electrode capacitor Cn is also reduced.

In the present embodiment, the terminal-to-terminal voltage VD of the positive electrode capacitor Cp is a voltage higher than the output voltage Vd of the variable constant voltage circuit 44, and the magnitude of the terminal-to-terminal voltage −VD of the negative electrode capacitor Cn is larger than the magnitude of the output voltage −Vd of the variable constant voltage circuit 50.

That is, each time the pulser circuit 54 outputs the push wave transmission signal TX to the ultrasound oscillator 56 over a plurality of times, the positive electrode power supply 40 charges the positive electrode capacitor Cp with the voltage higher than the output voltage of the variable constant voltage circuit 44 (constant voltage circuit). Similarly, each time the pulser circuit 54 outputs the push wave transmission signal TX to the ultrasound oscillator 56 over a plurality of times, the negative electrode power supply 46 charges the negative electrode capacitor Cn with the voltage higher than the output voltage of the variable constant voltage circuit 50 (constant voltage circuit).

As shown in FIG. 4, each time the continuous positive/negative pulse voltage 200 is applied to the ultrasound oscillator 56, the magnitudes of the terminal-to-terminal voltage VD of the positive electrode capacitor Cp and the terminal-to-terminal voltage −VD of the negative electrode capacitor Cn are reduced. On the other hand, the variable constant voltage circuit 44 maintains the voltage Vd between the positive electrode terminal Tp of the pulser circuit 54 and the ground conductor within a predetermined range above and below the target voltage Vd* determined by the controller 20. Similarly, the variable constant voltage circuit 50 maintains the voltage −Vd between the negative electrode terminal Tn of the pulser circuit 54 and the ground conductor within a predetermined range above and below the target voltage −Vd* determined by the controller 20. Here, the term “within a predetermined range” is determined as, for example, ±5% or less based on the predetermined target voltage Vd*.

As a result, the amplitude of the continuous positive/negative pulse voltage 200 applied to the ultrasound oscillator 56 is maintained within the predetermined range. Therefore, a droop phenomenon occurring in the push wave transmitted from the ultrasound oscillator 56 to the subject 32 is suppressed, or the droop phenomenon does not occur in the push wave transmitted to the subject 32, and the energy is maintained at a sufficient magnitude. Further, the electrostatic capacitance of the positive electrode capacitor Cp and the negative electrode capacitor Cn for setting the amplitude of the continuous positive/negative pulse voltage 200 within the predetermined range is smaller than that in a case in which the variable constant voltage circuit 44 and the variable constant voltage circuit 50 are not used. As a result, the power supply device for push wave generation 52 is reduced in size.

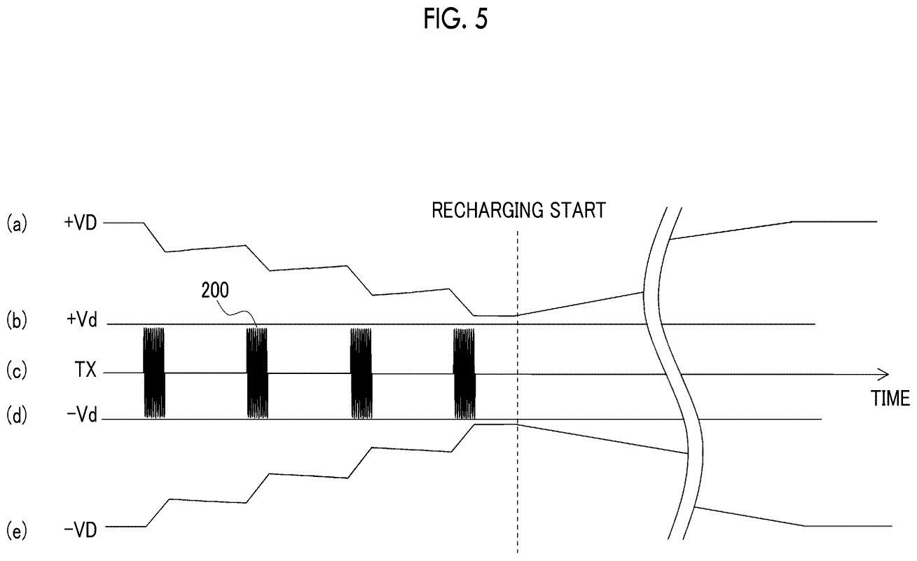

FIG. 5 shows each voltage in a case in which the control according to a second embodiment is executed in the power supply device for push wave generation 52. That is, (a) to (e) of FIG. 5 respectively show the time waveforms of the terminal-to-terminal voltage VD of the positive electrode capacitor Cp, the voltage Vd of the positive electrode terminal Tp of the pulser circuit 54, the push wave transmission signal TX applied from the pulser circuit 54 to the ultrasound oscillator 56, the voltage −Vd of the negative electrode terminal Tn of the pulser circuit 54, and the terminal-to-terminal voltage −VD of the negative electrode capacitor Cn. The terminal-to-terminal voltage VD of the positive electrode capacitor Cp is set to be higher than the voltage Vd of the positive electrode terminal Tp of the pulser circuit 54, and the magnitude of the terminal-to-terminal voltage −VD of the negative electrode capacitor Cn is set to be larger than the magnitude of the voltage −Vd of the negative electrode terminal Tn of the pulser circuit 54.

The controller 20 controls the variable constant voltage circuit 44 such that the voltage applied to the positive electrode terminal Tp of the pulser circuit 54 is maintained within the predetermined range above and below the target voltage Vd* determined by the controller 20. Similarly, the controller 20 controls the variable constant voltage circuit 50 such that the voltage applied to the negative electrode terminal Tn of the pulser circuit 54 is maintained within the predetermined range above and below the target voltage −Vd* determined by the controller 20.

On the other hand, each time the continuous transmission operation in which the push waves are continuously transmitted to the subject 32 over a plurality of times is repeated M+1 times in a time having a length of M times the transmission period T, the controller 20 controls the positive electrode power supply 40, the constant current circuit 42, the negative electrode power supply 46, and the constant current circuit 48 such that the magnitudes of the terminal-to-terminal voltage VD of the positive electrode capacitor Cp and the terminal-to-terminal voltage −VD of the negative electrode capacitor Cn are larger than that of the output voltage Vd. Here, M may be an integer of 2 or more.

The variable constant voltage circuit 44 maintains the voltage Vd between the positive electrode terminal Tp of the pulser circuit 54 and the ground conductor within the predetermined range above and below the target voltage Vd* determined by the controller 20. Similarly, the variable constant voltage circuit 50 maintains the voltage −Vd between the negative electrode terminal Tn of the pulser circuit 54 and the ground conductor within the predetermined range above and below the target voltage −Vd* determined by the controller 20.

During a period in which the positive electrode capacitor Cp and the negative electrode capacitor Cn are charged first and then the positive electrode capacitor Cp and the negative electrode capacitor Cn are charged next, the continuous transmission operation is repeated M+1 times, and the terminal-to-terminal voltage of each of the positive electrode capacitor Cp and the negative electrode capacitor Cn is sequentially decreased. However, the voltages of the positive electrode terminal Tp and the negative electrode terminal Tn of the pulser circuit 54 are maintained within the predetermined range by the variable constant voltage circuit 44 and the variable constant voltage circuit 50.

With the control according to the present embodiment, even in a case in which the plurality of continuous transmission operations are performed, the energy of the push wave transmitted from the ultrasound oscillator 56 to the subject 32 is maintained at a sufficient magnitude.

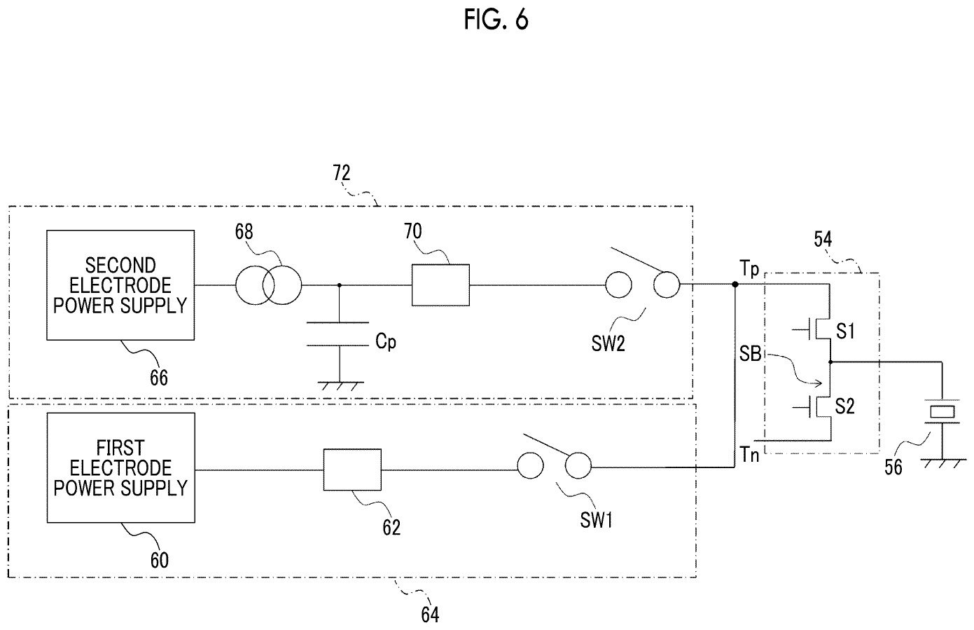

FIG. 6 shows a power supply device for push wave generation 72 according to a third embodiment, along with a power supply device for an analysis operation 64, the pulser circuit 54, and the ultrasound oscillator 56. The power supply device for push wave generation 72 is a power supply device for providing a basic type ultrasound diagnostic apparatus that does not have a function of performing the elasticity analysis (elasticity analysis function) with the elasticity analysis function at a later time. The power supply device for push wave generation 72 is attachable to and detachable from the basic type ultrasound diagnostic apparatus. The power supply device for an analysis operation 64 is installed in the basic type ultrasound diagnostic apparatus. In a case in which the ultrasound diagnostic apparatus 100 shown in FIG. 1 is the basic type ultrasound diagnostic apparatus, the power supply device for an analysis operation 64 may be formed in the power supply unit 34.

As in the first embodiment and the second embodiment, the power supply device for push wave generation 72 and the power supply device for an analysis operation 64 according to the third embodiment are controlled by the controller 20. For the sake of simplicity of the description, FIG. 6 shows only the components connected to the positive electrode terminal Tp of the pulser circuit 54, but the power supply device for push wave generation 72 also includes the components connected to the negative electrode terminal Tn of the pulser circuit 54. The components (not shown) connected to the negative electrode terminal Tn of the pulser circuit 54 have a configuration in which the polarity of the voltage and the direction in which the current flows are reversed with respect to the components connected to the positive electrode terminal Tp of the pulser circuit 54, and have the same configuration as the components on the positive electrode terminal Tp side. A configuration in which complementary components in which the voltage and the current have reverse polarities are connected to the positive electrode terminal Tp and the negative electrode terminal Tn of the pulser circuit 54 is also the same for a fourth embodiment described below.

The power supply device for an analysis operation 64 is a power supply device for causing the ultrasound oscillator 56 to generate the analysis ultrasound. The power supply device for an analysis operation 64 comprises a first positive electrode power supply 60, a first variable constant voltage circuit 62, and a first switch SW1. The first positive electrode power supply 60 outputs a power supply voltage to the first variable constant voltage circuit 62.

The first switch SW1 is turned on or off in accordance with the control of the controller 20. The first switch SW1 may be turned on or off in accordance with an operation of a user depending on an operation status. In a case in which the first switch SW1 is turned on, the first variable constant voltage circuit 62 outputs a voltage having a magnitude according to the control of the controller 20 to the positive electrode terminal Tp of the pulser circuit 54. The pulser circuit 54 is switched in accordance with the control of the controller 20 and outputs a pulse voltage for generating the analysis ultrasound to the ultrasound oscillator 56 as an analysis transmission signal. The ultrasound oscillator 56 transmits the analysis ultrasound in response to the analysis transmission signal output from the pulser circuit 54. The basic type ultrasound diagnostic apparatus generates image data representing the ultrasound image, such as an image showing an elasticity analysis result or a B-mode image, based on the analysis ultrasound reflected in the subject 32 and received by the ultrasound oscillator 56. In a case in which the first switch SW1 is turned off, the first variable constant voltage circuit 62 is electrically disconnected from the positive electrode terminal Tp of the pulser circuit 54.

The power supply device for push wave generation 72 comprises a second positive electrode power supply 66, a constant current circuit 68, the positive electrode capacitor Cp, a second variable constant voltage circuit 70, and a second switch SW2. The operations of the second positive electrode power supply 66, the constant current circuit 68, the positive electrode capacitor Cp, and the second variable constant voltage circuit 70 are the same as the operations of the positive electrode power supply 40, the constant current circuit 42, the positive electrode capacitor Cp, and the variable constant voltage circuit 44 provided in the power supply device for push wave generation 52 according to the first embodiment.

The second switch SW2 is turned on or off in accordance with the control of the controller 20. The second switch SW2 may be turned on or off in accordance with the operation of the user depending on the operation status. In a case in which the second switch SW2 is turned on, the second variable constant voltage circuit 70 outputs the voltage Vd having a magnitude according to the control of the controller 20 to the positive electrode terminal Tp of the pulser circuit 54. The pulser circuit 54 is switched in accordance with the control of the controller 20 and outputs the positive/negative pulse voltage for generating the push wave as the push wave transmission signal to the ultrasound oscillator 56. The ultrasound oscillator 56 transmits the push wave in response to the positive/negative pulse voltage output from the pulser circuit 54. In a case in which the second switch SW2 is turned off, the second variable constant voltage circuit 70 is electrically disconnected from the positive electrode terminal Tp of the pulser circuit 54.

In a case in which the power supply device for push wave generation 72 is not attached to the basic type ultrasound diagnostic apparatus, the first switch SW1 is turned on in accordance with the operation of the user or the control of the controller 20. In a case in which the elasticity analysis is performed in a case in which the power supply device for push wave generation 72 is attached to the basic type ultrasound diagnostic apparatus, the controller 20 may control the first switch SW1 and the second switch SW2 as follows. That is, in a case in which the ultrasound oscillator 56 transmits the push wave, the controller 20 turns off the first switch SW1 and turns on the second switch SW2. In a case in which the ultrasound oscillator 56 transmits the tracking wave or the ultrasound for generating the B-mode image, the controller 20 turns on the first switch SW1 and turns off the second switch SW2.

As described above, the power supply device for push wave generation 72 according to the present embodiment is attachably and detachably attached to the basic type ultrasound diagnostic apparatus. As one embodiment, the power supply device for push wave generation 72 comprises the first positive electrode power supply 60 (first power supply), the ultrasound oscillator 56, the pulser circuit 54 (pulse generation circuit), and a main power supply path for transmitting the power from the first positive electrode power supply 60 to the pulser circuit 54 in a case in which the ultrasound image is acquired. The main power supply path includes the first variable constant voltage circuit 62 and the first switch SW1 (main power supply path current cutoff/adjuster).

The power supply device for push wave generation 72 comprises the second positive electrode power supply 66 (second power supply) that outputs the supply power to the pulser circuit 54, the positive electrode capacitor Cp that accumulates the supply power, the second variable constant voltage circuit 70 (constant voltage circuit) provided between the positive electrode capacitor Cp and the pulser circuit 54, and the second switch SW2 as the current cutoff/adjuster provided between the second variable constant voltage circuit 70 and the pulser circuit 54.

In the power supply device for push wave generation 72, a terminal (downstream end) of the second switch SW2 on the pulser circuit 54 side is connected to the main power supply path. The pulser circuit 54 outputs the push wave transmission signal to the ultrasound oscillator 56 by using the power transmitted via the second switch SW2, and the ultrasound oscillator 56 transmits the push wave to the biological tissue of the subject 32 by using the push wave transmission signal.

The power supply device for push wave generation 72 according to the third embodiment is attached to the basic type ultrasound diagnostic apparatus, so that the function of the elasticity analysis is installed in the basic type ultrasound diagnostic apparatus at a later time. Therefore, it is easy to develop a product in response to the demand of the user.

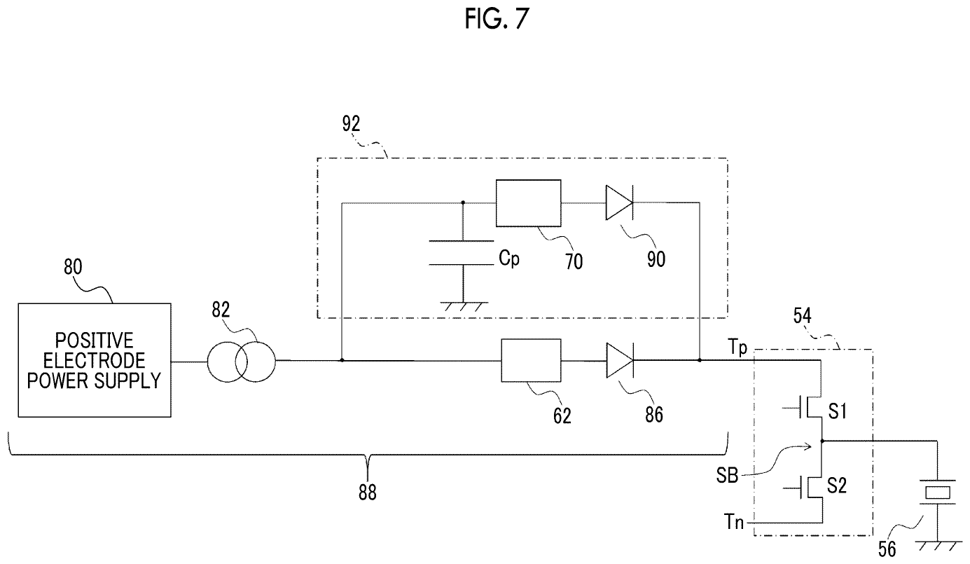

FIG. 7 shows an external power supply circuit 92 according to the fourth embodiment, along with a power supply device for an analysis operation 88, the pulser circuit 54, and the ultrasound oscillator 56. The external power supply circuit 92 is a circuit that provides the basic type ultrasound diagnostic apparatus with the elasticity analysis function, and is attachable to and detachable from the power supply device for an analysis operation 88 provided in the basic type ultrasound diagnostic apparatus. Although only the components connected to the positive electrode terminal Tp of the pulser circuit 54 are shown in FIG. 7, as in the third embodiment, complementary components in which the voltage and the current have reverse polarities are connected to the positive electrode terminal Tp and the negative electrode terminal Tn of the pulser circuit 54.

The power supply device for an analysis operation 88 comprises a positive electrode power supply 80, a constant current circuit 82, the first variable constant voltage circuit 62, and a first rectifier 86. The positive electrode power supply 80 outputs a predetermined voltage to the constant current circuit 82. The constant current circuit 82 outputs a current to the first variable constant voltage circuit 62 and the external power supply circuit 92 based on the voltage output from the positive electrode power supply 80.

The first variable constant voltage circuit 62 outputs a voltage according to the control of the controller 20 to the positive electrode terminal Tp of the pulser circuit 54 via the first rectifier 86. The first rectifier 86 limits directions of the current flowing through the first variable constant voltage circuit 62 and the pulser circuit 54 to one direction. The pulser circuit 54 is switched in accordance with the control of the controller 20 and outputs the pulse voltage for generating the analysis ultrasound to the ultrasound oscillator 56 as the analysis transmission signal. The ultrasound oscillator 56 transmits the analysis ultrasound in accordance with the pulse voltage output from the pulser circuit 54.

The external power supply circuit 92 is connected to the positive electrode terminal Tp of the pulser circuit 54 along with the power supply device for an analysis operation 88. The external power supply circuit 92 comprises the positive electrode capacitor Cp, the second variable constant voltage circuit 70, and a second rectifier 90. The positive electrode capacitor Cp is charged with a current flowing from the constant current circuit 82 to the external power supply circuit 92. The second variable constant voltage circuit 70 outputs a voltage to the positive electrode terminal Tp of the pulser circuit 54 via the second rectifier 90 based on the terminal-to-terminal voltage of the positive electrode capacitor Cp in accordance with the control of the controller 20. The second rectifier 90 limits directions of the current flowing through the second variable constant voltage circuit 70 and the pulser circuit 54 in one direction by the same operation as that of the first rectifier 86.

The pulser circuit 54 is switched in accordance with the control of the controller 20 and outputs the positive/negative pulse voltage for generating the push wave as the push wave transmission signal to the ultrasound oscillator 56. The ultrasound oscillator 56 transmits the push wave in response to the positive/negative pulse voltage output from the pulser circuit 54.

As described above, the external power supply circuit 92 (power supply device for push wave generation) according to the fourth embodiment is attachably and detachably attached to the basic type ultrasound diagnostic apparatus comprising the positive electrode power supply 80 (power supply), the ultrasound oscillator 56, the pulser circuit 54 (pulse generation circuit) to which the ultrasound oscillator 56 is connected, and the main power supply path that transmits the power from the positive electrode power supply 80 to the pulser circuit 54 in a case in which the ultrasound image is acquired. The main power supply path includes the first variable constant voltage circuit 62 and the first rectifier 86 (main power supply path current cutoff/adjuster).

The external power supply circuit 92 comprises the positive electrode capacitor Cp that accumulates the power output from the positive electrode power supply 80 and supplied to the pulser circuit 54, the second variable constant voltage circuit 70 (constant voltage circuit) that is provided between the positive electrode capacitor Cp and the pulser circuit 54, and the second rectifier 90 as the current cutoff/adjuster provided between the second variable constant voltage circuit 70 and the pulser circuit 54. A terminal (downstream end) of the second rectifier 90 on the pulser circuit 54 side is connected to the main power supply path. The pulser circuit 54 outputs the push wave transmission signal to the ultrasound oscillator 56 by using the power transmitted via the second rectifier 90, and the ultrasound oscillator 56 transmits the push wave to the biological tissue by using the push wave transmission signal.

In the fourth embodiment, the external power supply circuit 92 is provided with the second rectifier 90. Therefore, in a case in which the power is supplied from the positive electrode power supply 80 to the pulser circuit 54 via the constant current circuit 82, the first variable constant voltage circuit 62, and the first rectifier 86, the inflow of the current to the positive electrode capacitor Cp or the second variable constant voltage circuit 70 is blocked. In addition, the first rectifier 86 is provided between the first variable constant voltage circuit 62 and the pulser circuit 54. Therefore, in a case in which the power is supplied from the positive electrode power supply 80 to the pulser circuit 54 via the constant current circuit 82 and the external power supply circuit 92, the inflow of the current to the first variable constant voltage circuit 62 is blocked.

In addition, in the fourth embodiment, the first switch SW1 and the second switch SW2 used in the third embodiment are not provided. Therefore, the processing of mutually switching between the operation of causing the ultrasound oscillator 56 to transmit the analysis ultrasound and the operation of causing the ultrasound oscillator 56 to transmit the push wave is simplified, and the processing executed by the controller 20 is simplified.

Further, by attaching the external power supply circuit 92 to the basic type ultrasound diagnostic apparatus, the basic type ultrasound diagnostic apparatus is installed with the function of the elasticity analysis at a later time. Therefore, it is easy to develop the product in response to the demand of the user.

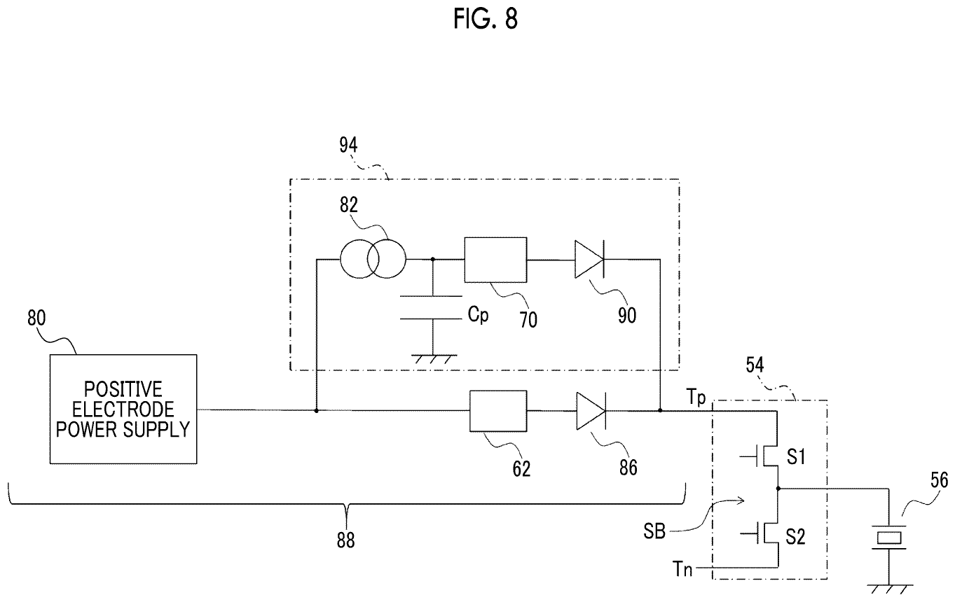

FIG. 8 shows an external power supply circuit 94 according to a modification example of the fourth embodiment, along with the power supply device for an analysis operation 88, the pulser circuit 54, and the ultrasound oscillator 56. This modification example is an example in which the position of the constant current circuit 82 is changed. That is, in the configuration shown in FIG. 7, the constant current circuit 82 is provided between the connection point between the external power supply circuit 92 and the first variable constant voltage circuit 62, and the positive electrode power supply 80, whereas in the configuration shown in FIG. 8, the constant current circuit 82 is provided between the connection point between the external power supply circuit 94 and the first variable constant voltage circuit 62, and the positive electrode capacitor Cp. The reason why such a modification can be made is that the constant current circuit 82 is intended to quickly charge the positive electrode capacitor Cp.

According to the modification example shown in FIG. 8, the same effect as the effect of the configuration shown in FIG. 7 can be obtained. That is, the processing of mutually switching between the operation of causing the ultrasound oscillator 56 to transmit the analysis ultrasound and the operation of causing the ultrasound oscillator 56 to transmit the push wave is simplified, and the processing executed by the controller 20 is simplified. Further, by attaching the external power supply circuit 94 to the basic type ultrasound diagnostic apparatus, the basic type ultrasound diagnostic apparatus is installed with the function of the elasticity analysis at a later time. Therefore, it is easy to develop the product in response to the demand of the user.

What is claimed is:

1. A power supply device for push wave generation comprising:
a power supply that outputs power supplied to a pulse generation circuit to which an ultrasound oscillator is connected;
a capacitor that accumulates the power supplied from the power supply; and
a constant voltage circuit that is provided between the capacitor and the pulse generation circuit,
wherein the pulse generation circuit outputs a transmission signal to the ultrasound oscillator, and
the ultrasound oscillator transmits a push wave in response to the transmission signal.

2. The power supply device for push wave generation according to claim 1,
wherein, each time the pulse generation circuit outputs the transmission signal to the ultrasound oscillator over a plurality of times, the power supply charges the capacitor with a voltage higher than an output voltage of the constant voltage circuit.

3. A power supply device for push wave generation being attachably and detachably attached to an ultrasound diagnostic apparatus including a first power supply, an ultrasound oscillator, a pulse generation circuit to which the ultrasound oscillator is connected, and a main power supply path that transmits power from the first power supply to the pulse generation circuit in a case in which an ultrasound image is acquired, the power supply device for push wave generation comprising:
a second power supply that outputs supply power to the pulse generation circuit;
a capacitor that accumulates the supply power;
a constant voltage circuit that is provided between the capacitor and the pulse generation circuit; and
a current cutoff/adjuster that is provided between the constant voltage circuit and the pulse generation circuit,
wherein a downstream end of the current cutoff/adjuster is connected to the main power supply path, and
the pulse generation circuit outputs a transmission signal to the ultrasound oscillator by using the power transmitted via the current cutoff/adjuster, and the ultrasound oscillator transmits a push wave to a biological tissue by using the transmission signal.

4. The power supply device for push wave generation according to claim 3,
wherein the main power supply path includes a main power supply path current cutoff/adjuster that is provided between the first power supply and the pulse generation circuit, and
the downstream end of the current cutoff/adjuster is connected between the main power supply path current cutoff/adjuster and the pulse generation circuit.

5. A power supply device for push wave generation being attachably and detachably attached to an ultrasound diagnostic apparatus including a power supply, an ultrasound oscillator, a pulse generation circuit to which the ultrasound oscillator is connected, and a main power supply path that transmits power from the power supply to the pulse generation circuit in a case in which an ultrasound image is acquired, the power supply device for push wave generation comprising:
a capacitor that accumulates the power output from the power supply and supplied to the pulse generation circuit;
a constant voltage circuit that is provided between the capacitor and the pulse generation circuit; and
a current cutoff/adjuster that is provided between the constant voltage circuit and the pulse generation circuit,
wherein a downstream end of the current cutoff/adjuster is connected to the main power supply path, and
the pulse generation circuit outputs a transmission signal to the ultrasound oscillator by using the power transmitted via the current cutoff/adjuster, and the ultrasound oscillator transmits a push wave to a biological tissue by using the transmission signal.

6. The power supply device for push wave generation according to claim 5,
wherein the main power supply path includes a main power supply path current cutoff/adjuster that is provided between the power supply and the pulse generation circuit, and
the downstream end of the current cutoff/adjuster is connected between the main power supply path current cutoff/adjuster and the pulse generation circuit.
